(19) United States
(12) Patent Application Publication
(10) Pub. No.: US 2019/0295694 A1
(43) Pub. Date: Sep. 26, 2019

(54) METHODS AND SYSTEMS FOR A HEALTHCARE PROVIDER SEARCH

(71) Applicant: Cambia Health Solutions, Inc., Portland, OR (US)

(72) Inventors: Cristopher Holm, Portland, OR (US); William S. McKinney, Portland, OR (US); Margret Mellon, Portland, OR (US)

(21) Appl. No.: 16/357,032

(22) Filed: Mar. 18, 2019

Related U.S. Application Data
(60) Provisional application No. 62/647,586, filed on Mar. 23, 2018.

(51) Int. Cl. G16H 10/20; G06F 16/9535

(52) U.S. Cl. CPC G16H 10/20; G16H 80/00; G06F 16/9535

(57) ABSTRACT

Systems and methods for searching for a healthcare provider based on an automatically-identified search context are provided. In one embodiment, a method comprises receiving, from a user, a search query, identifying one or more search contexts in a structured dictionary of medical terminology that at least partially matches the search query, calculating similarity scores for each of the one or more search contexts, selecting at least one search context of the one or more search contexts based on the calculated scores, performing a search for healthcare providers corresponding to the at least one search context, and displaying, to the user, results of the search including at least one healthcare provider. In this way, the search results may only include relevant results, so users may not be overwhelmed by a large volume of marginally-relevant search results.

### CROSS REFERENCE TO RELATED APPLICATIONS

The present application claims priority to U.S. Provisional Application No. 62/647,586, entitled “METHODS AND SYSTEMS FOR A HEALTHCARE PROVIDER SEARCH”, and filed on Mar. 23, 2018. The entire contents of the above-listed application are hereby incorporated by reference for all purposes.

### BACKGROUND AND SUMMARY

Users seeking medical care or treatment may wish to select a particular physician or healthcare provider to visit to receive the care or treatment. Factors that may be considered when trying to select a particular provider may include costs, location, specialty, quality ratings, and so on. To that end, users may use a web-based search engine to search a directory of healthcare providers. However, users may not know what search terms to use and therefore may have difficulty finding appropriate providers. For example, a user with foot pain may simply search “foot doctor” instead of “podiatrist,” and a typical search engine will likely return a list of providers with names similar to “foot” but specialize in a field other than podiatry.

Furthermore, typical search engines are designed to return results that include anything remotely relevant to the search query. Therefore, even if a user inputs an appropriate query, the search results may include many irrelevant results, thereby diluting the usefulness of the search results. Similarly, search engines do not recognize mixed-element search criteria (e.g., a search for a name plus a specialty).

The inventors have recognized the above issues and have devised several approaches to address them. In particular, systems and methods for searching for a healthcare provider based on an automatically-identified search context are provided. In one embodiment, a method comprises receiving, from a user, a search query, determining one or more search contexts in a structured dictionary of medical terminology that at least partially matches the search query, calculating scores for each of the one or more search contexts, selecting at least one search context of the one or more search contexts based on the calculated scores, performing a search for healthcare providers corresponding to the at least one search context, and displaying, to the user, results of the search including at least one healthcare provider. In this way, the search results may only include relevant results, so users may not be overwhelmed by a large volume of marginally-relevant search results.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE FIGURES

FIG. 1 illustrates an overview of an exemplary computing environment according to an embodiment.

FIG. 2 shows an overview of an exemplary arrangement of software modules for a vertical search engine for finding healthcare providers according to an embodiment.

FIG. 3 shows a high-level flow chart illustrating an example method for searching for a healthcare provider according to an embodiment.

FIG. 4 shows a high-level flow chart illustrating an example method for scoring a search context according to an embodiment.

FIG. 5 shows a high-level flow chart illustrating an example method for selecting a search context according to an embodiment.

FIGS. 6-8 show example user interfaces for a vertical search engine for finding healthcare providers according to an embodiment.

FIG. 9 shows a high-level flow chart illustrating an example method for searching for a healthcare provider according to an embodiment.

### DETAILED DESCRIPTION

The present description relates to systems and methods of searching for a healthcare provider. In particular, systems and methods for searching for a healthcare provider based on an automatically-identified search context are provided.

The systems and methods described herein provide a “vertical” search engine that focuses on a particular segment of content and applies subject-specific assumptions about relevance and relationships when generating a set of search results. In contrast, more generalized “horizontal” search engines query a broader set of information and are not structured to apply domain-specific rules when evaluating records for relevance as potential search results.

The systems and methods described herein provide the most relevant search results by inferring the intention behind each user's search. First, the query entered by the user is compared against the structured, searchable data available. The search algorithm supports searches against provider names, organization names, provider types, areas of expertise, specialties, and specialty synonyms or keywords, each of which contains a known set of searchable terms. If the user's input looks like a specialty, then the algorithm assumes the search is only for that specialty. If the user's input looks like a facility name, then the algorithm assumes the search is only for facilities by that name. In this way, the search results only include relevant results.

The systems and methods model dictionaries of known searchable data objects: specialties, areas of focus, keywords, and names. To determine the intent of a user's search, the algorithm looks for specialty and keyword exact matches in the dictionaries first, then names, then close specialties, then close last names, and once the algorithm finds a matching result, the algorithm performs the search based on that match. When the user misspells content, the algorithm recommends the proper spelling of medical terms if appropriate.

The systems and methods described herein provide a number of benefits. Users are less overwhelmed by a large volume of marginally-relevant search results. Users are provided with alternate search contexts in the event that the algorithm did not correctly interpret the search intent on the first pass. Simultaneous searches across multiple information types are supported. Expert domain knowledge of healthcare or data available in the system is not necessary for a user to find a relevant provider.

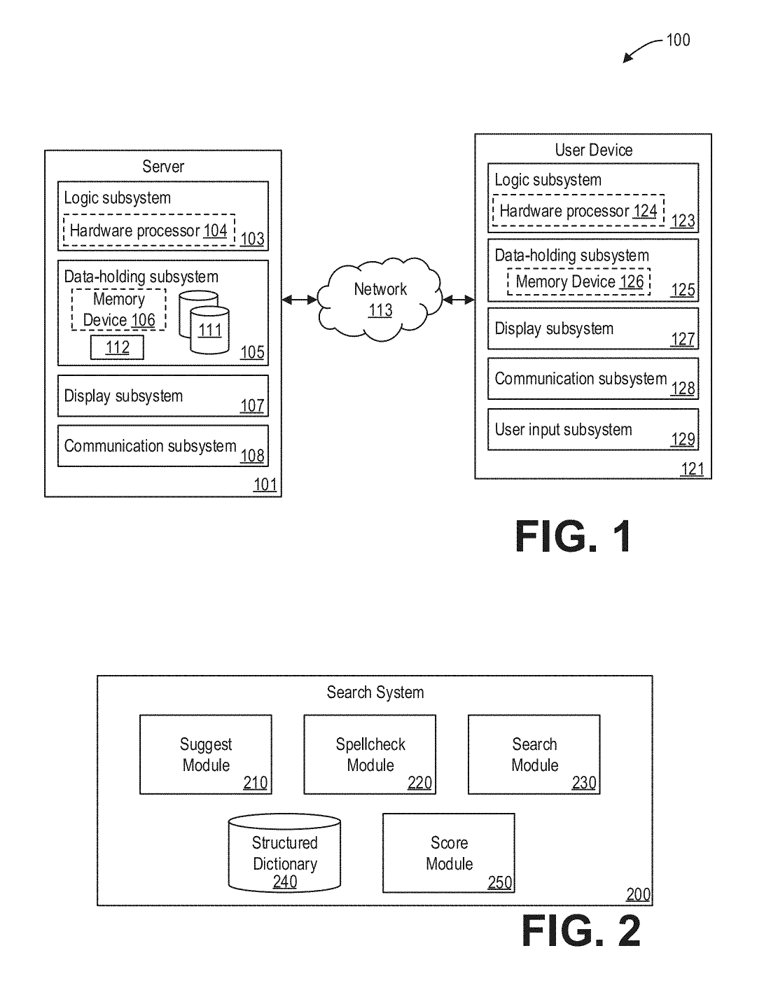

FIG. 1 illustrates an example computing environment 100 in accordance with the current disclosure. In particular, computing environment 100 includes a server 101, a plurality of user devices or client systems 121, and a network 113. However, not all of the components illustrated may be required to practice the invention. Variations in the arrangement and type of the components may be made without departing from the spirit or scope of the invention.

Server 101 may be a computing device configured to determine search contexts based on query input to narrow a search for a healthcare provider. In different embodiments, server 101 may take the form of a mainframe computer, server computer, desktop computer, laptop computer, tablet computer, home entertainment computer, network computing device, mobile computing device, mobile communication device, gaming device, etc.

Server 101 includes a logic subsystem 103 and a data-holding subsystem 105. Server 101 may optionally include a display subsystem 107, communication subsystem 108, and/or other components not shown in FIG. 1. For example, server 101 may also optionally include user input devices such as keyboards, mice, game controllers, cameras, microphones, and/or touch screens.

Logic subsystem 103 may include one or more physical devices configured to execute one or more instructions. For example, logic subsystem 103 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 103 may include one or more hardware processors 104 that are configured to execute software instructions. Additionally or alternatively, the logic subsystem 103 may include one or more hardware or firmware logic machines configured to execute hardware or firmware instructions. Processors of the logic subsystem 103 may be single or multi-core, and the programs executed thereon may be configured for parallel or distributed processing. The logic subsystem 103 may optionally include individual components that are distributed throughout two or more devices, which may be remotely located and/or configured for coordinated processing. One or more aspects of the logic subsystem 103 may be virtualized and executed by remotely accessible networked computing devices configured in a cloud computing configuration.

Data-holding subsystem 105 may include one or more physical, non-transitory memory devices 106 configured to hold data and/or instructions executable by the logic subsystem 103 to implement the herein described methods and processes. When such methods and processes are implemented, the state of data-holding subsystem 105 may be transformed (for example, to hold different data).

Data-holding subsystem 105 may include removable media and/or built-in devices. Data-holding subsystem 105 may include optical memory (for example, CD, DVD, HD-DVD, Blu-Ray Disc, etc.), and/or magnetic memory devices (for example, hard drive disk, floppy disk drive, tape drive, MRAM, etc.), and the like. Data-holding subsystem 105 may include devices with one or more of the following characteristics: volatile, nonvolatile, dynamic, static, read/write, read-only, random access, sequential access, location addressable, file addressable, and content addressable. In some embodiments, logic subsystem 103 and data-holding subsystem 105 may be integrated into one or more common devices, such as an application-specific integrated circuit or a system on a chip.

It is to be appreciated that data-holding subsystem 105 includes one or more physical, non-transitory devices. In contrast, in some embodiments aspects of the instructions described herein may be propagated in a transitory fashion by a pure signal (for example, an electromagnetic signal) that is not held by a physical device for at least a finite duration. Furthermore, data and/or other forms of information pertaining to the present disclosure may be propagated by a pure signal.

As discussed further herein, the data-holding subsystem 105 stores a search system in the form of executable instructions 112. When the executable instructions 112 are executed by one or more hardware processors of the logic subsystem 103 to run the search system on the one or more hardware processors of the logic subsystem 103, the logic subsystem 103 is operable to perform a search of one or more databases including one or more provider databases 111. A provider database 111 stores a list or a table of health care providers. The provider database 111 may further include additional metadata for each provider, including but not limited to provider specialties, location information (e.g., address, business hours, accessibility), availability (e.g., whether or not the provider is accepting new patients), descriptive information (e.g., gender, one or more photographs, a biographical description, language(s) spoken) and so on.

When included, display subsystem 107 may be used to present a visual representation of data held by data-holding subsystem 105. As the herein described methods and processes change the data held by the data-holding subsystem 105, and thus transform the state of the data-holding subsystem 105, the state of display subsystem 107 may likewise be transformed to visually represent changes in the underlying data. Display subsystem 107 may include one or more display devices utilizing virtually any type of technology. Such display devices may be combined with logic subsystem 103 and/or data-holding subsystem 105 in a shared enclosure, or such display devices may be peripheral display devices.

When included, communication subsystem 108 may be configured to communicatively couple server 101 with one or more other computing devices, such as user device 121. Communication subsystem 108 may include wired and/or wireless communication devices compatible with one or more different communication protocols. As non-limiting examples, communication subsystem 108 may be configured for communication via a wireless telephone network, a wireless local area network, a wired local area network, a wireless wide area network, a wired wide area network, etc. In some embodiments, communication subsystem 108 may allow server 101 to send and/or receive messages to and/or from other devices via a network such as the public Internet. For example, communication subsystem 108 may communicatively couple server 101 with user device 121 via network 113. In some examples, network 113 may be the public Internet. In other examples, network 113 may be regarded as a private network connection and may include, for example, a virtual private network or an encryption or other security mechanism employed over the public Internet.

Further, the server 101 provides a network service that is accessible to a plurality of users through a plurality of client systems 121 communicatively coupled to the server 101 via a network 113. As such, computing environment 100 may include one or more devices operated by users, such as user device 121. User device 121 may be any computing device configured to access a network such as network 113, including but not limited to a personal computer, a laptop, a smartphone, a tablet, and the like. While one user device or client system 121 are shown, it should be appreciated that any number of user devices may be communicatively coupled to the server 101 via the network 113.

User device 121 includes a logic subsystem 123 and a data-holding subsystem 125. User device 121 may optionally include a display subsystem 127, communication subsystem 128, and/or other components not shown in FIG. 1.

Logic subsystem 123 may include one or more physical devices configured to execute one or more instructions. For example, logic subsystem 123 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 123 may include one or more hardware processors 124 that are configured to execute software instructions. Additionally or alternatively, the logic subsystem 123 may include one or more hardware or firmware logic machines configured to execute hardware or firmware instructions. Processors of the logic subsystem 123 may be single or multi-core, and the programs executed thereon may be configured for parallel or distributed processing. The logic subsystem 123 may optionally include individual components that are distributed throughout two or more devices, which may be remotely located and/or configured for coordinated processing. One or more aspects of the logic subsystem 123 may be virtualized and executed by remotely accessible networking computing devices configured in a cloud computing configuration.

Data-holding subsystem 125 may include one or more physical, non-transitory memory devices 126 configured to hold data and/or instructions executable by the logic subsystem 123 to implement the herein described methods and processes. When such methods and processes are implemented, the state of data-holding subsystem 125 may be transformed (for example, to hold different data).

Data-holding subsystem 125 may include removable media and/or built-in devices. Data-holding subsystem 125 may include optical memory (for example, CD, DVD, HD-DVD, Blu-Ray Disc, etc.), and/or magnetic memory devices (for example, hard drive disk, floppy disk drive, tape drive, MRAM, etc.), and the like. Data-holding subsystem 125 may include devices with one or more of the following characteristics: volatile, nonvolatile, dynamic, static, read/write, read-only, random access, sequential access, location addressable, file addressable, and content addressable. In some embodiments, logic subsystem 123 and data-holding subsystem 125 may be integrated into one or more common devices, such as an application-specific integrated circuit or a system on a chip.

When included, display subsystem 127 may be used to present a visual representation of data held by data-holding subsystem 125. As the herein described methods and processes change the data held by the data-holding subsystem 125, and thus transform the state of the data-holding subsystem 125, the state of display subsystem 127 may likewise be transformed to visually represent changes in the underlying data. Display subsystem 127 may include one or more display devices utilizing virtually any type of technology. Such display devices may be combined with logic subsystem 123 and/or data-holding subsystem 125 in a shared enclosure, or such display devices may be peripheral display devices.

When included, communication subsystem 128 may be configured to communicatively couple user device 121 with one or more other computing devices, such as server 101. Communication subsystem 128 may include wired and/or wireless communication devices compatible with one or more different communication protocols. As non-limiting examples, communication subsystem 128 may be configured for communication via a wireless telephone network, a wireless local area network, a wired local area network, a wireless wide area network, a wired wide area network, etc. In some embodiments, communication subsystem 128 may allow user device 101 to send and/or receive messages to and/or from other devices, such as server 101, via a network 113 such as the public Internet.

User device 121 may further include a user input subsystem 129 comprising user input devices such as keyboards, mice, game controllers, cameras, microphones, and/or touch screens. A user of user device 121 may input a search query, for example, via user input subsystem 129. As discussed further herein, user device 121 may stream, via communication subsystem 128, user input received via the user input subsystem 129 to the server 101 in real time over the network 113. In this way, the server 101 may automatically suggest a search query based on the user input before the user formally submits a query to the server 101.

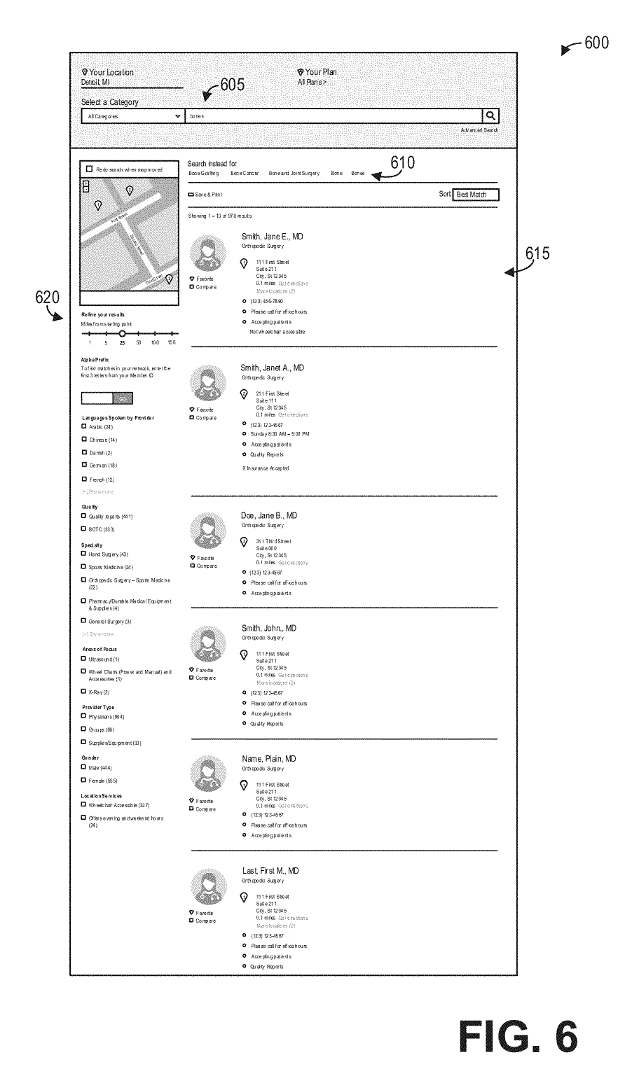

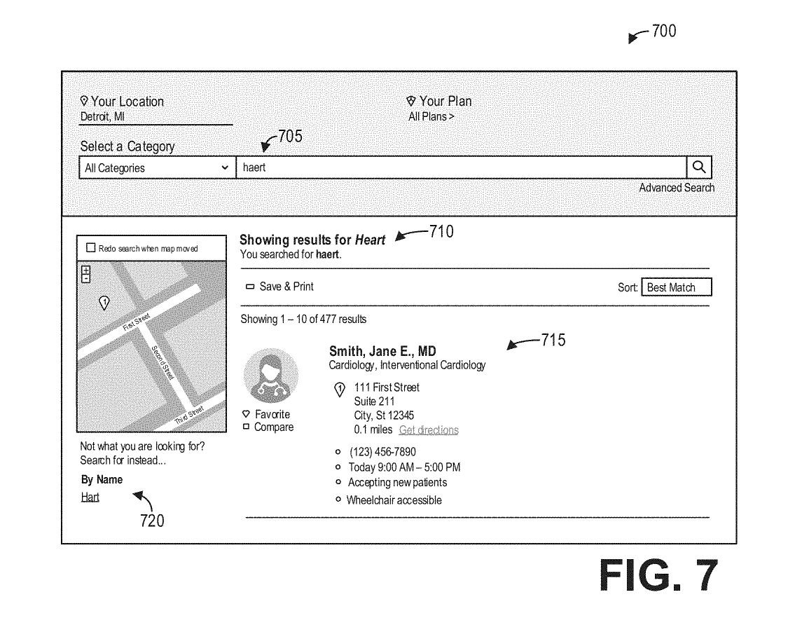

Thus server 101 and user device 121 may each represent computing devices which may generally include any device that is configured to perform computation and that is capable of sending and receiving data communications by way of one or more wired and/or wireless communication interfaces. Such devices may be configured to communicate using any of a variety of network protocols. For example, user device 121 may be configured to execute a browser application that employs HTTP to request information from server 101 and then displays the retrieved information to a user on a display. Example interfaces that may be delivered to user device 121 from server 101 in such a manner and displayed, for example, on display subsystem 127 are described further herein and with regard to FIGS. 6-8.

FIG. 2 shows an overview of an exemplary arrangement of software modules for a search system 200 for implementing a vertical search engine for finding healthcare providers. In particular, the search system 200 may include a suggest module 210, a spellcheck module 220, a search module 230, a structured dictionary 240, and a score module 250. Search system 200 may, for example, be included as executable instructions 112 stored in a non-transitory memory device 106 of the data-holding subsystem 105 of server 101.

Suggest module 210 generates suggested input based on received query input. To that end, suggest module 210 receives a search query, compares the search query to relevant search terms to determine whether the search query corresponds to a known term, and outputs identified terms that may correspond to the search query. The suggest module 210 does not assume that the received search query is a complete query, and therefore does not necessarily treat a word in a received query as a fully-formed word. For example, if the query “pedia” is input to suggest module 210, suggest module 210 may interpret “pedia” as a word that is either fully formed or that is a fragment for the beginning of a word. Suggest module 210 may compare the query “pedia” to known terms in a structured dictionary 240, and identify “pediatrics” and “pediatric cardiology” as possible suggestions because both terms include “pedia.” Suggest module 210 may therefore output “pediatrics” and “pediatric cardiology” as suggestions, which in turn may be displayed to the user as autocomplete suggestions.

The suggest module 210 may be accessed via a suggest application programming interface (API) which receives the query input and outputs suggestions based on the query input. It should be appreciated that the use of suggest module 210 for generating autocomplete suggestions may be asynchronous such that the query input by a user via user device 121, for example, is transmitted in real-time to server 101, wherein suggest module 210 determines and outputs suggestions based on the query back to the user device 121, wherein the suggestions are displayed to the user as they continue inputting the query. Thus, as a user types “pedia” into a search interface, the suggestions “pediatrics” and “pediatric cardiology” may be displayed to the user as autocomplete suggestions before the user formally submits the query. In this way, suggestions may be presented to a user after the user types only a few characters, with the suggestions getting closer to the user's intention as typing continues.

As another example, if “back pai” is input to the suggest module 210, the suggest module 210 interprets “back pai” as two words, both of which are either fully formed, or both fragments, or one of each. Consequently, suggest module 210 may suggest “back pain,” “back ache,” “pain management” and so on as autocomplete suggestions for “back pai”.

The spellcheck module 220 checks the spelling of input. When provider data is loaded, each specialty and synonym attached to every provider is added to a pool of known terms, which are stored in the structured dictionary 240. If the term follows a pattern that should be stemmed, its root and all derivative permutations are added to the pool of known terms. When a suggest operation is performed by suggest module 210, each word of the input in the request is examined by the spellcheck module 220 to see if it is a possible misspelling of the pool of known terms. To determine whether the input in the request includes a possible misspelling of the pool of known terms, the spellcheck module 220 calculates the similarity of the input word(s) with words in the pool of known terms. If the similarity score is above a certain threshold, the input word(s) may be considered misspelled, in which case the spellcheck module 220 may replace the input word with the term from the pool. Additionally, spellcheck module 220 performs word-breaking and word-combining corrections. An example of word-breaking is to transform input “familypractice” into “family practice.” An example of word-combining is to transform input “mammo gram” into “mammogram.”

Spellcheck module 220 also performs name spellchecking. Name spellchecking uses the same word-breaking and word-combining approach as specialties and synonyms. Then similarity comparisons are made on provider names.

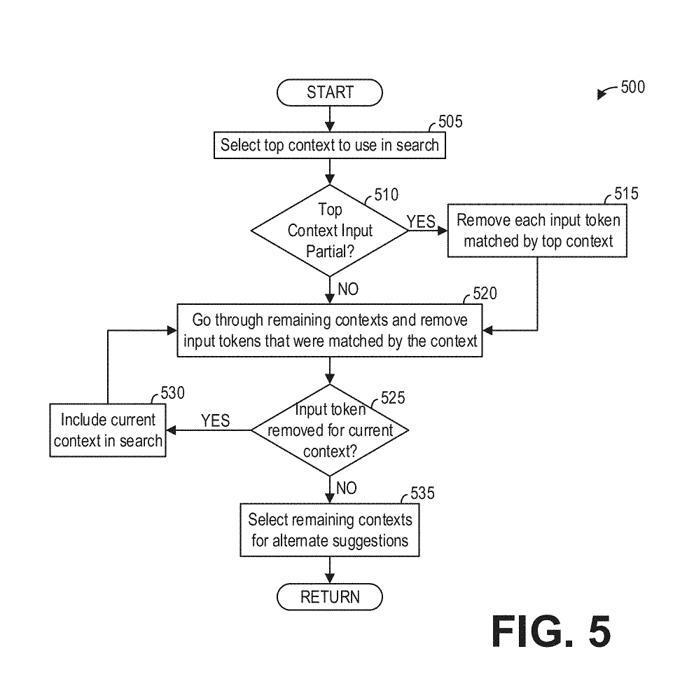

Search module 230 drives the search API which are responsible for performing specific searches on specific provider attributes. The search module 230 uses the results from the suggest module 210 to determine which suggestions best match the user's input and execute a particular search depending on which context was chosen as “best.” To that end, score module 250 calculates a score for each context or suggestion, as described further herein with regard to FIG. 5.

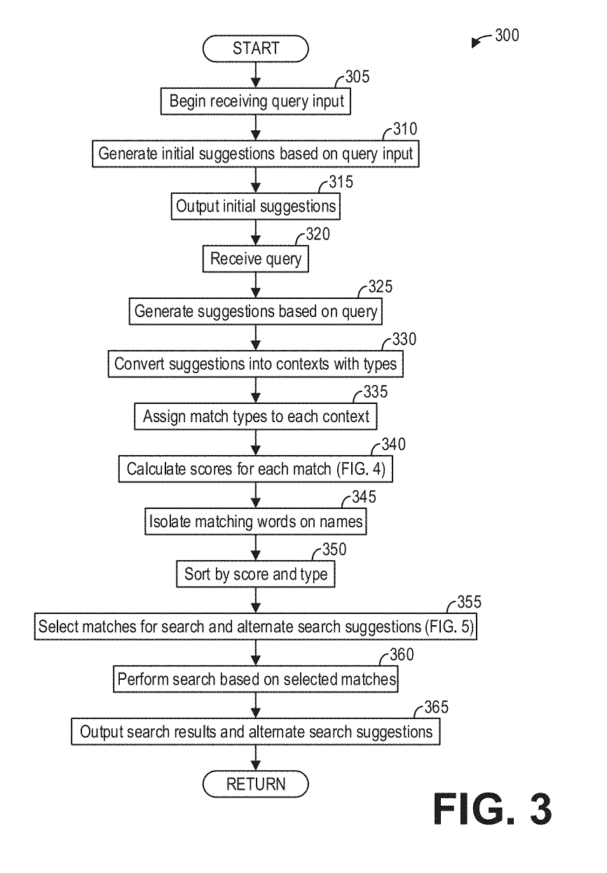

FIG. 3 shows a high-level flow chart illustrating an example method 300 for searching for a healthcare provider according to an embodiment. In particular, method 300 relates to automatically identifying a search context based on query input, and performing a search for a healthcare provider based on the identified search context. In this way, the results of the search may limited to relevant results. Method 300 is described with reference to the systems and components of FIGS. 1 and 2, though it should be appreciated that the method may be implemented with other systems and components without departing from the scope of the present disclosure. Method 300 may be stored as executable instructions in non-transitory memory 106, for example, on server 101 and may be executed by one or more hardware processors 104 of the logic subsystem 103.

Method 300 begins at 305. At 305, method 300 begins receiving query input. The query input comprises text input by a user that is not yet submitted as a query. Therefore, as a user of a user device, such as user device 121, enters text to form a search query, for example via user input subsystem 129, the user device 121 transmits the query input over a network 113 to the server 101. In some examples, the query input further comprises additional data aside from the text input by the user, such as location information (e.g., a geographic region for limiting the search), health care insurance information, and so on.

At 310, method 300 generates initial suggestions based on the query input. For example, as query input is received, method 300 compares the received query input to a structured dictionary of known terms and names to identify known terms and names that correspond to the received query input. To that end, method 300 may input the query input to the suggest module 210 to generate the initial suggestions. For example, the query input may include at least a portion of a word input by the user as well as a desired location region for searching, and so the suggest module 210 may at least generate initial suggestions according to the portion of the word. Further, in some examples, the suggest module 210 may limit suggestions according to the location information, such that the name of a provider outside of the desired location region for search is not output as a suggestion. At 315, method 300 outputs the initial suggestions or autocomplete suggestions. For example, the known terms and names identified at 310 are then displayed to a user as autocomplete suggestions. The user may select one of the autocomplete suggestions to submit as a query and/or continue manually inputting the query.

At 320, method 300 receives the query. The query comprises the text string submitted by the user. Furthermore, in some examples, the query further comprises the location information and/or health care plan information for limiting the search. Continuing at 325, method 300 generates suggestions based on the query. As an illustrative example, method 300 may input the query into the suggest module 210 to generate the suggestions based on the query.

Rather than displaying the suggestions to the user as autocomplete suggestions, as performed at 315, method 300 uses the suggestions generated by the suggest module 210 to determine the context of the search query and thus narrow the search. To that end, at 330, method 300 converts the suggestions into contexts with types. More specifically, method 300 converts each suggestion generated at 325 into a context with a type that characterizes the suggestion. The type may comprise, as illustrative and non-limiting examples: NAME (indicating that the suggestion is a name for any type of provider), PERSON_NAME (indicating names for only person providers), ORG_NAME (indicating names for non-person providers, i.e., organization providers), SPECIALTY (indicating specialties for any type of provider), PERSON_SPECIALTY (indicating specialties for person providers), ORG_SPECIALTY (indicating specialties for an organization provider), SYNONYM (indicating a specialty synonym or keyword for any type of provider), PERSON_SYNONYM (indicating specialty synonyms for person providers), ORG_SYNONYM (indicating specialty synonyms for organization providers), PROVIDER_SUBTYPE (indicating subtypes or a super-type of specialties for any type of provider), PERSON_PROVIDER_SUBTYPE (indicating subtypes for only person providers), ORG_PROVIDER_SUBTYPE (indicating subtypes for organization providers), CARE_GROUP (indicating a care group), PROVIDER_OFFERING (indicating offerings or particular services for any type of provider), PERSON_PROVIDER_OFFERING (indicating offerings for person providers), ORG_PROVIDER_OFFERING (indicating offerings for organization providers), NPI (indicating that the suggestion is a National Provider Identifier), and so on.

At 335, method 300 assigns a match type to each context. The match type indicates how well the context corresponds to the user's input. As an illustrative example, the match type may comprise an “input complete, context complete” (ICCC) match type, an “input complete, context partial” (ICCP) match type, an “input partial, context complete” (IPCC) match type, or an “input partial, context partial” (IPCP) match type. An ICCC match type indicates that all input words (of the query) and all context words (of the suggestion) match each other completely. For example, if the query comprises “back pain” while the suggestion or context comprises “back pain,” then the input exactly matches the context: therefore, the match type is ICCC. An ICCP match type indicates that all input words were matched but one or more context words were unmatched. For example, if the input is “pain” and the context is “pain management,” then all input words (“pain”) are matched by the context but the word “management” in the context is not matched by the input: therefore, the match type is ICCP. An IPCC match type indicates that all context words were matched but one or more input words were unmatched. For example, if the input is “intolerable back pain” and the context is “back pain,” then all context words are matched by the input but the word “intolerable” in the input is not matched by the context: therefore, the match type is IPCC. An IPCP match type indicates that at least one input word was matched with at least one context word, but both the input and the context include unmatched words. For example, if the input is “pain in the caboose” and the context is “pain management,” the word “pain” is matched in both the input and the context while the remainder of the input and the context are unmatched: therefore, the match type is IPCP.

At 340, method 300 calculates scores for each match between a context and the query. In one example, method 300 calculates a score for a match between a context and the query based on the total number of words in the context, the total number of words in the query, the total number of words in the context that matched a word in the query, the total number of words in the context that exactly matched a word in the query, and the similarity of a matching context word to a query word.

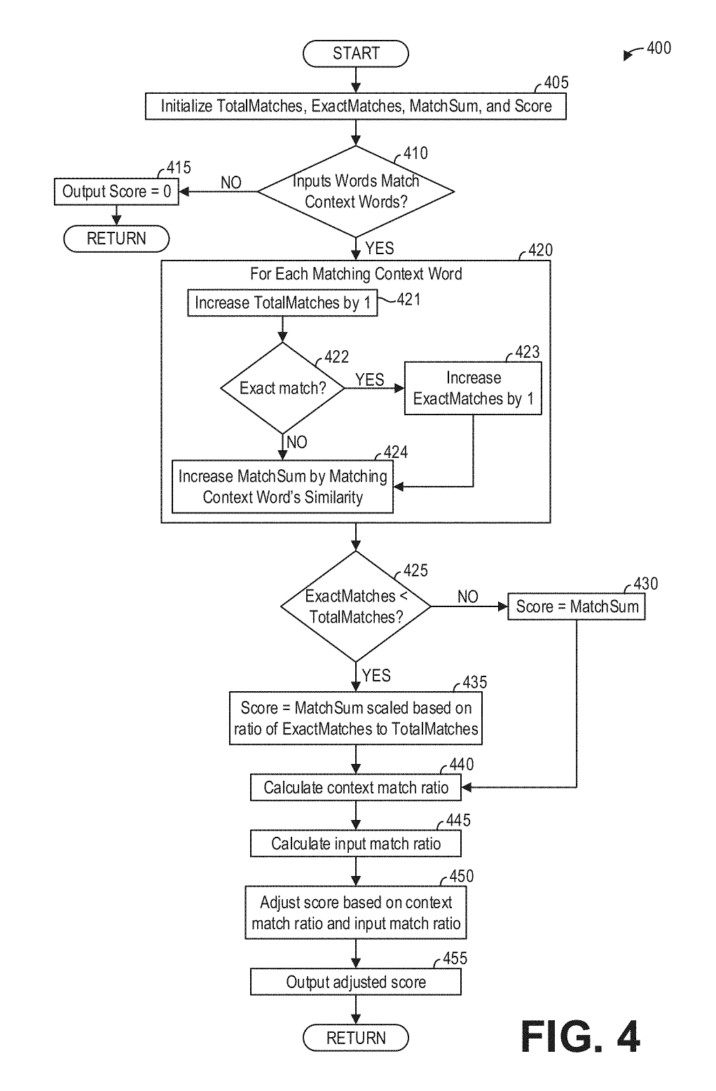

The score is calculated such that the score for an exact match between a context and a query is higher than an inexact match between a context and a query. For example, if the query is “emergency medicine,” the query may be matched with an “emergency medicine” context and an “internal medicine” context. The score for the match of the query with “emergency medicine” is higher than the score for the match of the query with “internal medicine.” An example method for calculating scores is described further herein with regard to FIG. 4.

After calculating the score for each match, method 300 continues to 345. At 345, method 300 isolates matching words on names. Method 300 treats matches between a query and a context with a NAME type differently than other types of matches. In particular, for a context with a NAME type, the context is modified after scoring to only include the words that were matched. In this way, the method eliminates duplicate matches. For example, if the query is “Smith” and there are two PERSON_NAME matches of “Smith, David” and “Smith, Roger,” both are equally valid; if selected for the search, the search should only be performed on the last name “Smith” without regard to other parts of the name. Therefore, method 300 reduces both contexts to “Smith” and discards one of the matches. Method 300 also isolates matching words on non-person (e.g., organization) names.

Continuing at 350, method 300 sorts the matches by score and type. Method 300 sorts the matches primarily by score. However, if some matches have the same score, an order of precedence is applied for matches with the same score. As an illustrative example, in the event of a tied score, the order of precedence for types may comprise: PROVIDER_OFFERING, SPECIALTY, SYNONYM, PERSON_NAME, ORG_NAME, PROVIDER_SUBTYPE, and CARE_GROUP.

At 355, method 300 selects matches for the search and alternate search suggestions. In one example, if the top match is input complete, method 300 selects that context for the search. Thus, for a single top match that is input complete, the search may comprise a single-context search. If the top match is input partial, method 300 attempts to find other matches that satisfy the input words that were not matched by the top match. When method 300 has matched all input tokens (i.e., each word in the query) or runs out of matches, method 300 selects the identified contexts for the search. Thus, for an input-partial top match, the search may comprise a multi-context search. A method for selecting matches and alternate search suggestions is described further herein with regard to FIG. 5.

At 360, method 300 performs a search based on the selected matches. As discussed above, the selected matches comprise one or more contexts that match the query. Performing the search based on the selected matches includes retrieving search results that correspond to the selected context(s). The results may be retrieved from a provider database, such as provider database 111, as a non-limiting example. Further, the search may be limited, for example, by the location information and/or the health care plan information. For example, the results retrieved from the provider database 111 may be limited to providers within the desired geographical region and/or who accept a specified insurance plan.

At 365, method 300 outputs search results and alternate search suggestions. In particular, method 300 outputs the search results and the alternate search suggestions to the user device 121 for display to the user via display subsystem 127. Example user interfaces that include example search results obtained according to method 300 are described further herein with regard to FIGS. 6-8. Method 300 then ends.

FIG. 4 shows a high-level flow chart illustrating an example method 400 for scoring a search context according to an embodiment. In particular, method 400 relates to calculating a score for a context that indicates how well the context matches with a query. Method 400 is described with reference to the systems and components of FIGS. 1 and 2, though it should be appreciated that the method may be implemented with other systems and components without departing from the scope of the present disclosure. Method 400 may be stored as executable instructions in non-transitory memory 106, for example, on server 101, and may be executed by one or more hardware processors 104 of the logic subsystem 103.

Method 400 begins at 405. At 405, method 400 initializes the variables TotalMatches, ExactMatches, MatchSum, and Score for the given context and query. Initializing the variables TotalMatches, ExactMatches, MatchSum, and Score comprises initially setting TotalMatches, ExactMatches, MatchSum, and Score equal to zero. As described further herein, the variable TotalMatches comprises a count of context words (i.e., words in a context) which match words in the input words (i.e., the one or more words of the query), the variable ExactMatches comprises a count of the context words which exactly matches an input word, the variable MatchSum comprises a measure of similarity between individual input words and individual context words, and the variable Score comprises a measure of similarity between the query and a context.

Continuing at 410, method 400 determines if any of the input words (i.e., the one or more words of the query) match any of the context words (i.e., the one or more words of the context). If none of the input words match any of the context words (“NO”), method 400 proceeds to 415. At 415, method 400 outputs a score of zero for the context. In this way, if the query does not match the context at all, the score for the match between the query and the context is zero. Method 400 then returns.

However, if at least one input word matches at least one context word (“YES”), method 400 proceeds to 420. At 420, method 400 iteratively calculates an initial score for each matching context word. Iteratively calculating an initial score for each matching context word includes increasing TotalMatches by one at 421. A context word may inexactly or exactly match an input word to qualify as a matching context word. At 422, method 400 determines if the matching context word is an exact match. If the matching context word is an exact match (“YES”), method 400 increases ExactMatches by one at 423. After increasing ExactMatches at 423, or if the matching context word is not an exact match (“NO”) at 422, method 400 increases MatchSum by the matching context word's similarity at 424. The similarity of a matching context word to an input word is a quantitative measure of how similar the matching context word is to the input word. To that end, the similarity may comprise a value ranging from zero to one, where a similarity of zero indicates that the context word and the input word are not similar at all, and wherein a similarity of one indicates that the context word and the input word are exactly the same. The similarity may be less than one but greater than zero, for example, if adjacent characters in a word are transposed (e.g., if the input word is “pedaitrics” which corresponds to “pediatrics” with the adjacent “a” and “i” letters transposed) or if a character in a word is different (e.g., if the input word is “pedeatrics” instead of “pediatrics” such that the input word has an “e” where it should have an “i”). Method 400 therefore calculates the similarity of the input word and the context word, and sets MatchSum equal to the calculated similarity.

After calculating an initial score for each matching context word at 420, method 400 proceeds to 425. At 425, method 400 determines if ExactMatches is less than TotalMatches. If the number of exact matches equals the number of total matches (“NO”), method 400 proceeds to 430. At 430, method 400 sets Score equal to MatchSum. Since each matching context word is an exact match with the input, the similarity between the input word and the context word calculated at 424 equals one for each matching context word, and therefore MatchSum comprises a value equal to the number of matching words (e.g., ExactMatches=TotalMatches=MatchSum). Therefore, the Score at 430 equals the number of matching words.

However, if the number of exact matches is less than the number of total matches (“YES”), method 400 proceeds to 435. At 435, method 400 sets Score equal to MatchSum scaled based on the ratio of ExactMatches to TotalMatches. In particular, method 400 drives down the Score based on how many matching words were not exact matches. To that end, method 400 multiplies the Score by a base multiplier but scaled to the ratio of words that were exact so that the boost is always in the range of [BaseMultiplier, 1.0) where BaseMultiplier is the base multiplier. As an illustrative and non-limiting example, the score may be calculated according to:

Score=MatchSum*(BaseMultiplier+(Ratio*(1−BaseMultiplier))),

where Ratio equals ExactMatches divided by TotalMatches. For example, if one word matched but was not exact (e.g., such that TotalMatches equals one and ExactMatches equals zero), then the Ratio is zero and the MatchSum is simply multiplied by the BaseMultiplier. If the BaseMultiplier is set to 0.3, for example, then the score is multiplied by 0.3. As another example, if two words matched wherein one match is exact and the other is not, then the Ratio is 0.5; if the BaseMultiplier is set to 0.3, for example, then the score is multiplied by (0.3+0.5*0.7)=0.65. As yet another example, if three words matched wherein two words are exact matches but one is not exact, then the Ratio is 2/3; for a BaseMultiplier of 0.3, the score is multiplied by (0.3+0.66*0.7)=0.767. AS another example, if three words matched wherein one word is an exact match and the other two are not exact, then the Ratio is 1/3; for a BaseMultiplier of 0.3, the score is multiplied by (0.3+0.33*0.7)=0.533.

After updating the Score at 430 or 435, method continues to 440. At 440, method 400 calculates the context match ratio. The context match ratio comprises the ratio of total matches (i.e., TotalMatches) to the total number of context words. In this way, the score may be adjusted based on how many words in the context were matched.

Similarly, at 445, method 400 calculates the input match ratio. The input match ratio comprises the ratio of total matches to the total number of input words. In this way, the score may be adjusted based on how many words in the input were matched.

Continuing at 450, method 400 adjusts the score based on the context match ratio and the input match ratio. For example, method 400 may multiply the Score obtained at 430 or 435 by the context match ratio and the input match ratio. Note that if the input exactly matches the context, the context match ratio and the input match ratio both equal one. Therefore, the Score equals the MatchSum obtained at 420, which comprises the number of words in the input.

After adjusting the score at 450, method 400 continues to 455. At 455, method 400 outputs the adjusted score for the similarity between the query and the context. Method 400 then returns. As method 400 relates to calculating a score indicating the similarity between a query and a single context, it should be appreciated that method 400 may be executed for a plurality of contexts as described herein.

FIG. 5 shows a high-level flow chart illustrating an example method 500 for selecting a search context according to an embodiment. Method 500 may comprise a subroutine of method 300. Method 500 is described with reference to the systems and components of FIGS. 1 and 2, though it should be appreciated that the method may be implemented with other systems and components without departing from the scope of the present disclosure. Method 500 may be stored as executable instructions in non-transitory memory 106, for example, on server 101 and may be executed by one or more hardware processors 104 of the logic subsystem 103.

Method 500 begins at 505. At 505, method 500 selects the top context to use in the search. The top context comprises the context with the highest score. Alternatively, if two contexts are tied for the highest score, the top context is selected from the two contexts based on the order of precedence for context type discussed hereinabove.

At 510, method 500 determines if the top context is input partial. As discussed hereinabove, a context is input partial if some but not all words in the input are matched against words in the context. If the top context is input partial (“YES”), method 500 proceeds to 515. At 515, method 500 removes each input token matched by the top context. An input token comprises a word of the input. For example, the input “pain management” may be tokenized into “pain” and “management” wherein each word is an input token. Therefore, after removing each input token matched by the top context, the remaining input tokens comprise the words in the input that were not matched by the top context. Method 500 then proceeds to 520.

Referring again to 510, if the top context is not input partial (“NO”), then the top context is input complete (i.e., each word of the input is matched with a word in the context), and method 500 proceeds directly to 520.

At 520, method 500 goes through the remaining contexts and removes input tokens that are matched by the context. Method 500 may iterate through the remaining contexts in order of score and type. For example, method 500 first removes input tokens that are matched by the second highest scoring context. At 525, method 500 determines if an input token is removed for the current context. If an input token is removed for the current context (“YES”), method 500 proceeds to 530. At 530, method 500 selects the current context to include in the search. Method 500 then loops back to 520 to continue evaluating the remaining contexts. Method 500 iterates through the remaining contexts until an input token is not removed (e.g., when there are no input tokens remaining). Once an input token is not removed for a current context at 525 (“NO”), method 500 proceeds to 535. At 535, method 500 selects the remaining contexts for alternate suggestions. Method 500 then returns.

As an illustrative example of how method 300, method 400, and method 500 may be used to perform a search for a healthcare provider, suppose a user submits “emergency medicine roberts” as a search query because they are searching for a person named “Roberts” who practices “Emergency Medicine.” The suggestions generated by the suggest module 210 may include, for example, “Emergency Medicine,” “Internal Medicine,” “Occupational Medicine,” “Roberts, Allison D,” and “Roberts Memorial Hospital.” These suggestions may be converted into contexts with a corresponding type. For example, the context “Emergency Medicine” has a “SPECIALTY” type, the context “Internal Medicine” has a “SPECIALTY” type, the context “Occupational Medicine” has a “SYNONYM” type, the context “Roberts, Allison D” has a “PERSON_NAME” type, and the context “Roberts Memorial Hospital” has an “ORG_NAME” type. A score is calculated for each context. For the context “Emergency Medicine,” the MatchSum is 2, the TotalMatches is 2, the total number of context words is 2, and the total number of input words is 3; therefore, the score of the match between “Emergency Medicine” and the input “emergency medicine roberts” is 1.334 according to the method 400 for calculating a score. Applying the same approach to the other contexts, the context “Internal Medicine” has a score of 0.167, the context “Occupational Medicine” has a score of 0.167, the context “Roberts, Allison D” has a score of 0.111, and the context “Roberts Memorial Hospital” has a score of 0.111. The top context is “Emergency Medicine” given that the score for this context is higher than the scores for the other contexts. In accordance with method 500, the context “Emergency Medicine” is thus selected for the search and the input tokens “emergency” and “medicine” are removed from the pool of input words used to identify matches, such that the remaining input token is “roberts.” When method 500 iterates through the remaining contexts, the method does not select the context “Internal Medicine” with the second highest score or the context “Occupational Medicine” with the third highest score because the input word “medicine” has already been assigned to a higher ranked match. The context “Roberts, Allison D” and the context “Roberts Memorial Hospital” have the same score but different types. Therefore, based on the order of precedence that prioritizes the “PERSON_NAME” type over the “ORG_NAME” type, the context “Roberts, Allison D” is selected for the search because the context matches the input token “roberts.” The input token “roberts” is therefore removed from the pool of input words, and so the context “Roberts Memorial Hospital” is not selected for the search because there are no more input words remaining to be matched. Further, by isolating the matching words on names, the contexts “Roberts, Allison D” and “Roberts Memorial Hospital” are both reduced to “Roberts.” Thus, the contexts selected for the search are “Emergency Medicine” and “Roberts” with respective types SPECIALTY and PERSON_NAME. A search is then performed that is limited to the selected contexts. The search therefore results with providers that have both the specialty of emergency medicine and the name Roberts.

It should be appreciated that there are two broad categories of match types: name-related (e.g., PERSON_NAME, ORG_NAME, and CARE_GROUP) and specialty-related (e.g., PROVIDER_OFFERING, SPECIALTY, SYNONYM, and PROVIDER_SUBTYPE). When multiple matches are selected from the same category, a search is performed in which resulting providers match at least one of the searched contexts. For example, the context “Internal Medicine” with a SPECIALTY type and the context “Occupational Medicine” with SYNONYM type would result in providers that have either the SPECIALTY type, the SYNONYM type, or both.

When multiple contexts are selected from different categories, a search is performed that results in providers that have at least one of the contexts from each category. For example, if the selected contexts are “Emergency Medicine” with a SPECIALTY type, “Occupational Medicine” with a SYNONYM type, and “Roberts” with a PERSON_NAME type, the search would result in providers that have the SPECIALTY or the SYNONYM type as well as the PERSON_NAME type.

Furthermore, as discussed hereinabove with regard to FIG. 5, contexts that are not selected for use in the search are offered as alternate search contexts. Further, if the search performed is a multi-context search, then each context is separately offered as an alternate search context. For example, if a user inputs “williams podiatric” but really meant to enter “williams pediatric,” the PERSON_NAME “Williams” and the SPECIALTY “Podiatry” are offered as separate searches that can be performed in isolation.

FIGS. 6-8 show example user interfaces for a vertical search engine for finding healthcare providers according to an embodiment. For example, FIG. 6 shows an example interface 600 depicting search results obtained in accordance with the methods described herein with regard to FIGS. 3-5 and FIG. 9. The interface 600 may be displayed to a user of a user device 121 via display subsystem 127, as a non-limiting example.

The interface 600 includes a search query input 605. As depicted, a user has input “bones” to the search query input 605. The top context selected is “Orthopedic Surgery,” and so the search results 615 comprise providers who specialize in orthopedic surgery, as depicted. A partial match on the “Bone” keyword has a higher order of precedence than the word “Bone” in clinic names or elsewhere. The interface 600 further displays alternate search contexts 610 that include “Bone Grafting,” “Bone Cancer,” “Bone and Joint Surgery,” “Bone,” and “Bones.” Additionally, the interface 600 includes a plurality of categories 620, unrelated or related to the search contexts, that allow the user to refine the results, for example based on languages spoken by the provider, quality, specialty, areas of focus, provider type, gender, location services, and distance from the user. Selections in the plurality of categories 620 may be submitted via the interface 600 along with the search query input 605 to limit the search accordingly.

It should be appreciated that in contrast with the present disclosure, a search for “bones” in a provider search engine that is not performed in accordance with the present disclosure may return a list of largely irrelevant fuzzy name matches, such as providers with the name “Jones” or “Mones.”

As another example, FIG. 7 shows an example interface 700 wherein a user has entered “haert” to the search query input 705. The spellcheck module 220 determines that the query is a misspelling of “heart” and therefore the suggest module 210 generates suggestions or contexts based on “heart” as the input. The interface 700 indicates 710 that “Heart” was used as the search query instead of “haert.” The search results 715 includes providers who specialize in Cardiology. An alternate search context of “Hart” with a PERSON_NAME type are displayed 720 by the interface 700. Thus, a search for a name that may also be a keyword or specialty may not initially return the name matches, but instead may return providers with specialties mapped to the “Heart” keyword.

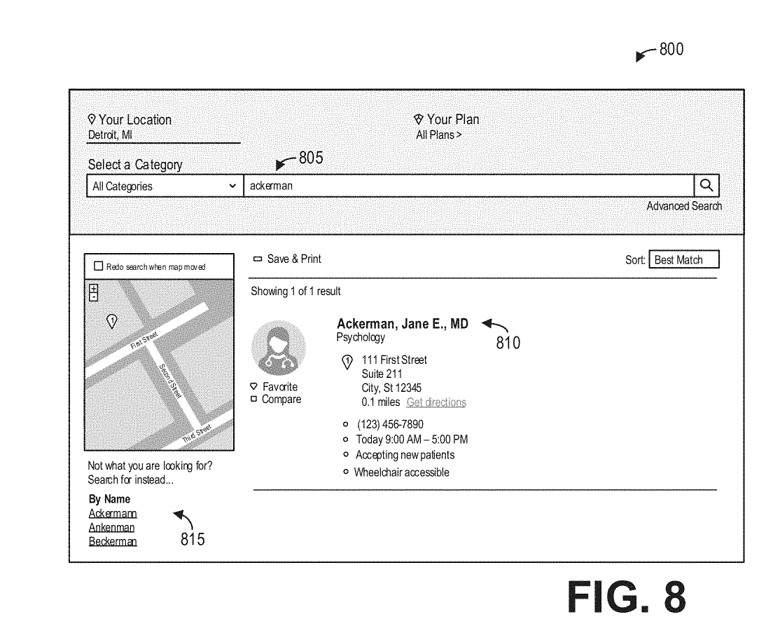

As yet another example, FIG. 8 shows an example interface 800 wherein a user has input “ackerman” to the search query input 805. The search result 810 includes a provider with the name “Ackerman,” while the alternate search contexts 815 include person names that are similar to “ackerman” and thus may be considered close enough to possibly be misspellings: “Ackermann,” “Ankenman,” and “Beckerman.” Thus, searches for names return only the closest matches in the results.

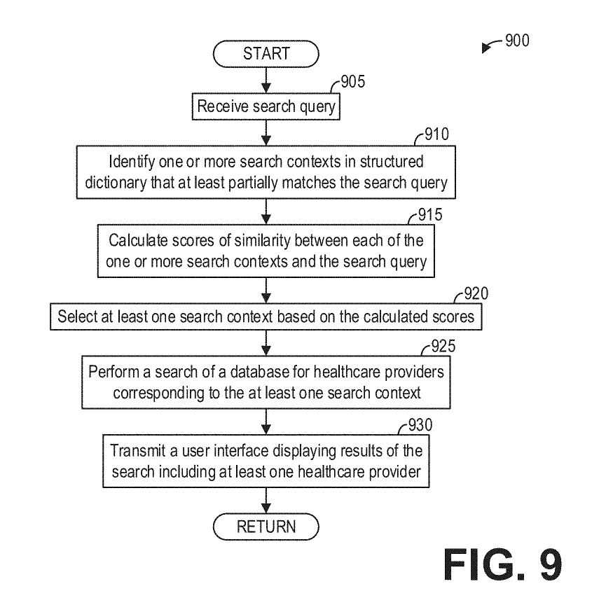

FIG. 9 shows another high-level flow chart illustrating an example method 900 for searching for a healthcare provider according to an embodiment. Method 900 is described with reference to the systems and components of FIGS. 1 and 2, though it should be appreciated that the method may be implemented with other systems and components without departing from the scope of the present disclosure. Method 900 may be stored as executable instructions in non-transitory memory 106, for example, on server 101 and may be executed by one or more hardware processors 104 of the logic subsystem 103. That is, method 900 comprises a computer-implemented method performed by one or more hardware processors 104.

Method 900 begins at 905. At 905, method 900 receives, over a network 113 from a user device 121, a search query input by a user to the user device 121. At 910, method 900 identifies one or more search contexts in a structured dictionary of medical terminology, such as structure dictionary 240, which at least partially matches the search query. At 915, method 900 calculates scores of similarity between each of the one or more search contexts and the search query. At 920, method 900 selects at least one search context of the one or more search contexts based on the calculated scores. At 925, method 900 performs a search of a database for healthcare providers corresponding to the at least one search context. At 930, method 900 transmits, over the network 113 to the user device 121 for display to the user, a user interface displaying results of the search including at least one healthcare provider. Method 900 then returns.

The systems and methods for a healthcare provider search described herein provide various advantages over previous search systems. For example, for a search performed in accordance with the present disclosure, relevant search results are retrieved from a massive dataset include a large number of potential results, and the relevant search results are displayed while irrelevant search results are not displayed. In this way, a user may obtain desired search results without having to manually parse a large number of search results. Further, by identifying an appropriate search context prior to performing a search, relevant search results are obtained faster because the search is limited to the appropriate search context. The search does not compare the search query to every data entry in a massive dataset of healthcare providers. Irrelevant or marginally relevant search results may not be displayed, or may be displayed as alternative search contexts if the relevancy score of the corresponding search context is substantial enough.

Thus, systems and methods for a healthcare provider search are provided. In one embodiment, a computer-implemented method, the method performed by one or more hardware processors, comprises receiving, over a network from a user device, a search query input by a user to the user device, identifying one or more search contexts in a structured dictionary of medical terminology that at least partially matches the search query, calculating scores of similarity between each of the one or more search contexts and the search query, selecting at least one search context of the one or more search contexts based on the calculated scores, performing a search of a database for healthcare providers corresponding to the at least one search context, and transmitting, over the network to the user device for display to the user, a user interface displaying results of the search including at least one healthcare provider.

In a first example of the method, each of the one or more search contexts includes a context type, wherein the context type comprises a provider offering, a specialty, a synonym for the specialty, a person name, or an organization name. In a second example of the method optionally including the first example, selecting the at least one search context is further based on the context type. In a third example of the method optionally including one or more of the first and second examples, calculating scores for each of the one or more search contexts comprises, for a search context of the one or more search contexts, calculating a score for the search context based on a comparison of words in the search query to words in one or more search contexts. In a fourth example of the method optionally including one or more of the first through third examples, the method further comprises scaling the score for the search context based on a ratio of words in the search query matched to words in the search context. In a fifth example of the method optionally including one or more of the first through fourth examples, the method further comprises receiving query input from the user prior to receiving the search query, generating autocomplete suggestions based on the received query input, and transmitting the autocomplete suggestions over the network to the user device for display to the user. In a sixth example of the method optionally including one or more of the first through fifth examples, the method further comprises transmitting alternate search suggestions over the network to the user device for display in the user interface to the user, the alternate search suggestions comprising at least one search context of the one or more search contexts not selected for the search.

In another embodiment, a system comprises a hardware processor, and a non-transitory memory device coupled to the hardware processor and storing executable instructions that when executed cause the hardware processor to: receive, from a user device via a network, a search query input to the user device by a user; automatically determine one or more search contexts based on the search query; calculate a score for each of the one or more search contexts indicating similarity of each of the one or more search contexts to the search query; and perform a search in a database of health care providers for the search query, the search limited to a search context of the one or more search contexts with a highest score.

In a first example of the system, each of the one or more search contexts includes a context type, wherein the context type comprises a provider offering, a specialty, a synonym for the specialty, a person name, or an organization name, and wherein the executable instructions when executed further cause the hardware processor to sort calculated scores for the one or more search contexts based on the context type. In a second example of the system optionally including the first example, the non-transitory memory device stores a structured dictionary of known terms, wherein the non-transitory memory device further stores a suggest module configured to receive the search query and output the one or more search contexts by comparing the search query to the structured dictionary. In a third example of the system optionally including one or more of the first and second examples, calculating scores for each of the one or more search contexts comprises, for a search context of the one or more search contexts, calculating a score for the search context based on a comparison of words in the search query to words in one or more search contexts. In a fourth example of the system optionally including one or more of the first through third examples, the non-transitory memory device further stores a spellcheck module configured to correct spelling of one or more words in the search query. In a fifth example of the system optionally including one or more of the first through fourth examples, the executable instructions when executed further cause the hardware processor to transmit search results from the search to the user device for display to the user. In a sixth example of the system optionally including one or more of the first through fifth examples, the executable instructions when executed further cause the hardware processor to transmit a search context of the one or more search contexts not used for the search to the user device for display as an alternate search suggestion. In a seventh example of the system optionally including one or more of the first through sixth examples, the non-transitory memory device further stores a score module configured to calculate the score for each of the one or more search contexts.

In yet another embodiment, a computer-readable storage medium stores a program of instructions executable by a machine to perform a method, the method comprising: receiving, from a user device over a network, a search query; generating suggestions based on the search query; converting the suggestions into contexts with types; calculating scores for each context of the contexts; selecting a first context with a highest score for a search and a second context for an alternate search suggestion; searching a database based on the first context; and transmitting, over the network to the user device, search results from the search and the alternate search suggestion for display to a user.

In a first example of the computer-readable storage medium, the method further comprises determining that the first context does not exactly match the search query, and selecting a third context for the search if the third context includes a word of the search query that is not matched by the first context. In a second example of the computer-readable storage medium optionally including the first example, selecting the second context for the alternate search suggestion comprises selecting a context of the contexts remaining after selecting the first context and the third context. In a third example of the computer-readable storage medium optionally including one or more of the first and second examples, calculating scores for each context of the contexts comprises, for a context of the contexts, calculating a score for the context based on a comparison of words in the search query to words in the context. In a fourth example of the computer-readable storage medium optionally including one or more of the first through third examples, the suggestions are generated by comparing the search query to a structured dictionary of known terms.

In another representation, a method comprises receiving, from a user, a search query, determining one or more search contexts in a structured dictionary of medical terminology that at least partially matches the search query, calculating scores for each of the one or more search contexts, selecting at least one search context of the one or more search contexts based on the calculated scores, performing a search for healthcare providers corresponding to the at least one search context, and displaying, to the user, results of the search including at least one healthcare provider.

In a first example of the method, each of the one or more search contexts includes a context type, wherein the context type comprises a provider offering, a specialty, a synonym for the specialty, a person name, or an organization name. In a second example of the method optionally including the first example, selecting the at least one search context is further based on the context type. In a third example of the method optionally including one or more of the first and second examples, calculating scores for each of the one or more search contexts comprises, for a search context of the one or more search contexts, calculating a score for the search context based on a comparison of words in the search query to words in one or more search contexts. In a fourth example of the method optionally including one or more of the first through third examples, the method further comprises scaling the score for the search context based on a ratio of words in the search query matched to words in the search context. In a fifth example of the method optionally including one or more of the first through fourth examples, the method further comprises receiving query input from the user prior to receiving the search query, generating autocomplete suggestions based on the received query input, and displaying the autocomplete suggestions to the user. In a sixth example of the method optionally including one or more of the first through fifth examples, the method further comprises outputting alternate search suggestions to the user, the alternate search suggestions comprising at least one search context of the one or more search contexts not selected for the search.

In another representation, a system comprises a logic subsystem and a data-holding subsystem storing executable instructions that when executed cause the logic subsystem to: receive a search query from a user; automatically determine one or more search contexts based on the search query; calculate a score for each of the one or more search contexts; and perform a search for the search query, the search limited to a search context of the one or more search contexts with a highest score.

In a first example of the system, each of the one or more search contexts includes a context type, wherein the context type comprises a provider offering, a specialty, a synonym for the specialty, a person name, or an organization name, and wherein the executable instructions when executed further cause the logic subsystem to sort calculated scores for the one or more search contexts based on the context type. In a second example of the system optionally including the first example, the data-holding subsystem stores a structured dictionary of known terms, and the data-holding subsystem further stores a suggest module configured to receive the search query and output the one or more search contexts by comparing the search query to the structured dictionary. In a third example of the system optionally including one or more of the first and second examples, calculating scores for each of the one or more search contexts comprises, for a search context of the one or more search contexts, calculating a score for the search context based on a comparison of words in the search query to words in one or more search contexts. In a fourth example of the system optionally including one or more of the first through third examples, the data-holding subsystem further stores a spellcheck module configured to correct spelling of one or more words in the search query. In a fifth example of the system optionally including one or more of the first through fourth examples, the executable instructions when executed further cause the logic subsystem to transmit search results from the search to a user device for display to the user. In a sixth example of the system optionally including one or more of the first through fifth examples, the executable instructions when executed further cause the logic subsystem to transmit a search context of the one or more search contexts not used for the search to the user device for display as an alternate search suggestion. In a seventh example of the system optionally including one or more of the first through sixth examples, the data-holding subsystem further stores a score module configured to calculate the score for each of the one or more search contexts.

In yet another representation, a method comprises receiving a search query; generating suggestions based on the search query; converting the suggestions into contexts with types; calculating scores for each context of the contexts; selecting a first context with a highest score for a search and a second context for an alternate search suggestion; performing the search based on the first context; and displaying search results from the search and the alternate search suggestion.

In a first example of the method, the method further comprises determining that the first context does not exactly match the search query, and selecting a third context for the search if the third context includes a word of the search query that is not matched by the first context. In a second example of the method optionally including the first example, selecting the second context for the alternate search suggestion comprises selecting a context of the contexts remaining after selecting the first context and the third context. In a third example of the method optionally including one or more of the first and second examples, calculating scores for each context of the contexts comprises, for a context of the contexts, calculating a score for the context based on a comparison of words in the search query to words in the context. In a fourth example of the method optionally including one or more of the first through third examples, the suggestions are generated by comparing the search query to a structured dictionary of known terms.

It will be appreciated that the configurations and routines disclosed herein are exemplary in nature, and that these specific embodiments are not to be considered in a limiting sense, because numerous variations are possible. The subject matter of the present disclosure includes all novel and non-obvious combinations and sub-combinations of the various systems and configurations, and other features, functions, and/or properties disclosed herein.

The following claims particularly point out certain combinations and sub-combinations regarded as novel and nonobvious. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application.

Such claims, whether broader, narrower, equal, or different in scope to the original claims, are also regarded as included within the subject matter of the present disclosure.

1. A computer-implemented method, the method performed by one or more hardware processors, comprising:
receiving, over a network from a user device, a search query input by a user to the user device;
identifying one or more search contexts in a structured dictionary of medical terminology that at least partially matches the search query;
calculating scores of similarity between each of the one or more search contexts and the search query;
selecting at least one search context of the one or more search contexts based on the calculated scores;
performing a search of a database for healthcare providers corresponding to the at least one search context; and
transmitting, over the network to the user device for display to the user, a user interface displaying results of the search including at least one healthcare provider.

2. The method of claim 1, wherein each of the one or more search contexts includes a context type, wherein the context type comprises a provider offering, a specialty, a synonym for the specialty, a person name, or an organization name.

3. The method of claim 2, wherein selecting the at least one search context is further based on the context type.

4. The method of claim 1, wherein calculating scores for each of the one or more search contexts comprises, for a search context of the one or more search contexts, calculating a score for the search context based on a comparison of words in the search query to words in one or more search contexts.

5. The method of claim 4, further comprising scaling the score for the search context based on a ratio of words in the search query matched to words in the search context.

6. The method of claim 1, further comprising receiving query input from the user prior to receiving the search query, generating autocomplete suggestions based on the received query input, and transmitting the autocomplete suggestions over the network to the user device for display to the user.

7. The method of claim 1, further comprising transmitting, over the network to the user device, alternate search suggestions for display in the user interface to the user, the alternate search suggestions comprising at least one search context of the one or more search contexts not selected for the search.

8. A system, comprising:
a hardware processor; and
a non-transitory memory device coupled to the hardware processor and storing executable instructions that when executed cause the hardware processor to:
receive, from a user device via a network, a search query input to the user device by a user;
automatically determine one or more search contexts based on the search query;
calculate a score for each of the one or more search contexts indicating similarity of each of the one or more search contexts to the search query; and
perform a search in a database of health care providers for the search query, the search limited to a search context of the one or more search contexts with a highest score.

9. The system of claim 8, wherein each of the one or more search contexts includes a context type, wherein the context type comprises a provider offering, a specialty, a synonym for the specialty, a person name, or an organization name, and wherein the executable instructions when executed further cause the hardware processor to sort calculated scores for the one or more search contexts based on the context type.

10. The system of claim 8, wherein the non-transitory memory device stores a structured dictionary of known terms, wherein the non-transitory memory device further stores a suggest module configured to receive the search query and output the one or more search contexts by comparing the search query to the structured dictionary.

11. The system of claim 8, wherein calculating scores for each of the one or more search contexts comprises, for a search context of the one or more search contexts, calculating a score for the search context based on a comparison of words in the search query to words in one or more search contexts.

12. The system of claim 8, wherein the non-transitory memory device further stores a spellcheck module configured to correct spelling of one or more words in the search query.

13. The system of claim 8, wherein the executable instructions when executed further cause the hardware processor to transmit search results from the search to the user device for display to the user.

14. The system of claim 13, wherein the executable instructions when executed further cause the hardware processor to transmit a search context of the one or more search contexts not used for the search to the user device for display as an alternate search suggestion.

15. The system of claim 8, wherein the non-transitory memory device further stores a score module configured to calculate the score for each of the one or more search contexts.

16. A computer-readable storage medium storing a program of instructions executable by a machine to perform a method, the method comprising:
receiving, from a user device over a network, a search query;
generating suggestions based on the search query;
converting the suggestions into contexts with types;
calculating scores for each context of the contexts;
selecting a first context with a highest score for a search and a second context for an alternate search suggestion;
searching a database based on the first context; and
transmitting, over the network to the user device, search results from the search and the alternate search suggestion for display to a user.

17. The computer-readable storage medium of claim 16, wherein the method further comprises determining that the first context does not exactly match the search query, and selecting a third context for the search if the third context includes a word of the search query that is not matched by the first context.

18. The computer-readable storage medium of claim 17, wherein selecting the second context for the alternate search suggestion comprises selecting a context of the contexts remaining after selecting the first context and the third context.

19. The computer-readable storage medium of claim 16, wherein calculating scores for each context of the contexts comprises, for a context of the contexts, calculating a score for the context based on a comparison of words in the search query to words in the context.

20. The computer-readable storage medium of claim 16, wherein the suggestions are generated by comparing the search query to a structured dictionary of known terms.
